(19) United States
(12) Patent Application Publication
(10) Pub. No.: US 2017/0106157 A1
(43) Pub. Date: Apr. 20, 2017

(54) Medical Gas Alarm System

(71) Applicant: BeaconMedaes LLC, Rock Hill, SC (US)

(72) Inventors: Ray Wilson, Charlotte, NC (US); Ben Levine, Fort Mill, SC (US)

(73) Assignee: BeaconMedaes LLC, Rock Hill, SC (US)

(21) Appl. No.: 15/393,827

(22) Filed: Dec. 29, 2016

Related U.S. Application Data
(62) Division of application No. 14/693,970, filed on Apr. 23, 2015.
(60) Provisional application No. 61/983,170, filed on Apr. 23, 2014.

(51) Int. Cl. A61M 16/00; G08B 21/12; H04L 12/28

(52) U.S. Cl. CPC A61M 16/0051; H04L 12/2823; G08B 21/12; H04W 84/12

(57) ABSTRACT

A medical gas alarm systems and associated methods are disclosed. A method of monitoring the medical gas system includes the steps of monitoring a characteristic of a medical gas system using at least one monitoring instrument positioned in a medical gas supply network; generating and sending a particular signal from the monitoring instrument to a CPU when the characteristic measured by the monitoring instrument passes a predetermined threshold; generating a fault signal from the CPU when the CPU determines that a fault condition has occurred; retrieving a stored message from the CPU in response to the fault signal, and in which the stored message other than the fault or threshold condition monitored by the instrument; and sending the stored message from the CPU to an output at a medical gas alarm module.

### RELATED APPLICATION

This patent application is a divisional of Ser. No. 14/693,970 filed Apr. 23, 2015 for “Medical Gas Alarm System.”

### BACKGROUND

The present invention relates to medical gas systems, medical gas alarms and related instruments.

In the modern medical environment, a number of gases are directed from sources to intended locations and then used for various purposes. These typically include oxygen (O₂) nitrogen (N₂), as nitrous oxide (NO₂), and medical air, collectively referred to as piped medical gases and vacuum systems or “PMGVS.” Such systems also include the capability to pull a vacuum where necessary at specific locations, but driven from a central location.

For convenience, the term “hospital” is used herein, but it will be understood that the relevant requirements and particular problems apply to other medical facilities outside of hospitals such as (but not limited to) urgent care centers and specialized surgical facilities.

PMGVS, however, are typically maintained in one or more central (figuratively speaking) locations or “banks” in large cylinders that can supply these gases throughout a hospital. The relevant gases are carried from the bank to the destinations through a network of pipes, conduits, supply lines, and the like to deliver gas to (or pull a vacuum at) the specific locations. Overall systems typically include (at least) compressors, vacuum pumps, tanks and manifolds.

For a number of practical reasons, including safety, the network is often divided into zones, and subzones to help control, detect, and solve problems.

At a minimum, medical gases (and the equipment that supplies them) are required for patient treatment. In many cases the gases directly sustain patients' lives, and thus the loss of gas supply can be fatal. Based upon those requirements, both best practices and the regulatory oversight and encouragement of best practices, mandate that the movement of such gases within the hospital network must be carefully monitored and controlled. NFPA (National Fire Protection Association) 99 is an exemplary health care facilities code.

Accordingly, as part of best practices and related regulatory overlay, medical gas systems incorporate alarms which signal appropriately when a gas supply is reduced (e.g., as reflected by a change in pressure or other compromise) or completely interrupted.

As used herein, the term “medical gas system” refers to the equipment that provides medical gases throughout a hospital (or equivalent facility), including but not limited to tanks, compressors, pumps (including vacuum pumps) and manifolds. The term “medical gas alarm system” refers to any device or group of devices that measure or detect one or more physical characteristics of a gas or its identity, or any relevant status of any source or equipment, in the medical environment, and that produces some form of output based upon the detected item or value. A “system” can thus include alarms, processors, controls, switches and the like. A “Master Alarm Panel” monitors medical gas and vacuum source equipment and main pipelines (e.g., monitors open condition from source equipment or pressure switch on main pipeline). An “Area Alarm Panel” monitors medical gas and vacuum systems serving a specific area (e.g., monitors pressure transducer reading; processor determines fault condition). A “Combo Alarm Panel” combines features of a master alarm panel and an area alarm panel.

In most cases the alarm is connected mechanically or electrically or both to related items in the medical gas system and its gas distribution network. To the extent that the alarm is difficult to read or interpret, or provides ambiguous information, the alarm is less helpful. In the current state-of-the-art, medical gas alarms use buzzers for an audible alert, and generally only provide information about a given condition; e.g. the pressure for gas at a particular location or zone. To the extent an individual that is familiar with the facility and it's gases recognizes what a particular signal means, such sounds or numerals are, of course helpful. Nevertheless a signal that merely indicates that a gas has dropped below an alarm's threshold level, or even a signal that the condition of a gas or of its supply may have been compromised, may not provide all of the information that could be helpful or necessary in a given circumstance.

The current landscape also uses alarms that signals and gas sensors, which are helpful, but again must be interpreted (typically) by a knowledgeable person. When such alarms are connected to one another in a “master,” and “slave” relationship (i.e., two separate displays, but in which the second merely duplicates the output from the first) the respective circuit boards are wired directly to each other. Such a direct wired connection requires appropriate hardware and architectural accommodations.

As another current disadvantage, to the extent that alarms give textual information, they tend to be handwritten or printed labels that are limited by size and legibility.

Some newer alarms have the capability to collect and store data (for example in random access memory) and then produce the data on command. Conventionally, this requires an operator to visit each such memory equipped alarm board and collect logs from which appropriate reports can be generated.

As another factor, current alarms tend to lack any information other than the identity of a gas and it's particular pressure at a given time

As yet another problematic factor in the medical alarm environment, different national standards or practices operate using different types of networks. For example, in the United Kingdom, the two types of networks include “Medipoint” and “Shire.” The communication standards are different for each of these systems, and thus the entire alarm system of any sizable facility must be limited to one type of network and one type of alarm. Blended networks can present significant problems or functionally are simply impossible to connect.

Current conventional alarms also lack some of the updated display capabilities that are familiar to most lay persons in the form of smart phones, tablet computers, retail point-of-purchase electronics and the like.

Medical gas alarms are also, of course, used in most countries around the world, including many that do not use the English alphabet. From the standpoint of computing and digital memory, most words in English, and other Latin-based languages (e.g., French, Spanish) and even some non-Latin languages (e.g., German) can be formed from the 26 letters of the English alphabet. In some cases a few additional symbols such as umlauts and accents are helpful or necessary, but the number of these is relatively small. Furthermore, English-language characters are relatively small in terms of their memory requirements and usually an English letter can be stored with as little as one byte.

Non-English or non-Latin languages, however, can include much larger letter sets, or in some cases (e.g., Japanese and Chinese) large number of pictorial characters. Short simple messages of the type needed from a medical gas alarm are thus more difficult to produce in such languages because their complexity and the memory requirements to reproduce as visible output is much greater than for English alphabet languages.

Finally, many current alarms are inefficient with respect to the manner in which they can handle input and output signals including directing one input signal from a pressure transducer through many relay outputs, or many signal inputs from source equipment to a single relay output, or combinations of these functions.

### SUMMARY

In one aspect, the invention is a method of monitoring a medical gas system that includes the steps of monitoring a characteristic of the medical gas system using at least one monitoring instrument positioned in a medical gas supply network; generating and sending a particular signal from the monitoring instrument to a CPU when the characteristic measured by the monitoring instrument passes a predetermined threshold; generating a fault signal from the CPU when the CPU receives the particular threshold signal from the monitoring instrument; retrieving a stored message from the CPU in response to the fault signal, and in which the stored message other than the fault or threshold condition monitored by the instrument; and sending the stored message from the CPU to an output at a medical gas alarm module.

In another aspect the invention is the improvement in a medical gas alarm system that includes a gas sensor positioned or arranged to measure a characteristic of a medical gas in a medical gas supply network; a programmable monitor for displaying the gas characteristic from the gas sensor; and a 4-20 mA current loop between the gas sensor and the monitor for calibrating the desired output on the monitor using the 4-20 mA current range

In another aspect the invention is a medical gas alarm system that includes at least first and second (master-slave; primary-secondary) alarm stations; and an ethernet interface for each station. The first alarm station is connected to a gas monitoring instrument, and the first alarm station transmits information generated at or originally received at the first alarm station using Ethernet protocol over an available Ethernet network to the second alarm system. The second alarm station is connected to the first alarm station over the Ethernet network and the second alarm station displays the information from the first alarm station.

In another aspect the invention is the improvement in a medical gas alarm system that includes memory; graphic images stored in the memory; a display in communication with the memory; and a processor in communication with both the memory and the display.

In another aspect the invention is the improvement in a medical gas alarm system that includes a web server in the medical gas alarm; and a WiFi circuit in said alarm and in communication with the web server.

In another aspect the invention is a method of monitoring the status of a medical gas system that includes the steps of repeatedly measuring a characteristic of a medical gas system using a sensor positioned as part of the distribution network for a defined time interval; sending the characteristics measured by the sensor from the sensor to digital (or equivalent) memory in which the measurements can be stored and from which the indexed measurements can be retrieved; periodically retrieving groups of the stored and indexed measurements based upon a designated time interval (usually days) and sending the groups to a CPU; and generating a report from the CPU based on the retrieved groups in a form substantially compliant with a licensing or accreditation protocol.

In another aspect the invention is a medical gas alarm system that includes a gas sensor connected to a medical gas network; a medical gas alarm connected to the sensor; a CPU connected to the alarm; memory connected to the CPU; a human machine interface (HMI) connected to the CPU as output for the alarm and the gas sensor; and at least 30 permutations of text, color, line, and background designs that can be applied to the HMI and the output information the HMI provides based upon the sensor and the CPU.

In another aspect the invention is a medical gas alarm system that includes respective first and second communication interfaces from which a medical gas alarm can both receive and transmit. The first communication interface uses a different signal voltage and a different data rate than the second communication interface.

In another aspect the invention is a medical gas alarm system that includes a medical gas network; a sensor in the medical gas network; an alarm module in signal communication with the sensor; a CPU in the alarm module; and a touch screen display in the alarm module in an input/output relationship with the CPU for programming the CPU through the touch screen display.

In another aspect the invention is the improvement in a medical gas alarm system that includes a single input signal circuit in communication with source equipment; a processor in communication with the single input signal circuit; and at least two output relay circuits in communication with the processor.

In another aspect the invention is the improvement in a medical gas alarm system that includes a plurality of input signal circuits in communication with a respective plurality of source equipment; a processor in communication with the input signal circuits; and a single output relay circuit in communication with the processor. In another aspect, the alarm is an improvement on the communication of the condition(s) in default and the action to be taken by the staff, in accordance with the facility's operational and emergency plans.

The foregoing and other objects and advantages of the invention and the manner in which the same are accomplished will become clearer based on the followed detailed description taken in conjunction with the accompanying drawings.

### BRIEF DESCRIPTION OF THE DRAWINGS

FIG. 1 is a front perspective view of an alarm and in particular an alarm panel according to the invention.

FIG. 2 is a schematic diagram of the first embodiment of the invention.

FIG. 3 is a schematic diagram of a 4-20 mA loop in accordance with the present invention.

FIG. 4 is a schematic diagram of an embodiment of the invention that incorporates Ethernet capabilities.

FIG. 5 is a schematic diagram of an embodiment of the invention that can produce visual characters non-English on the display panel.

FIG. 6 is a schematic view of the alarm with Wi-Fi communication capabilities (among other capabilities).

FIG. 7 is a schematic diagram of an embodiment of the invention that produces some of the display aspects of FIG. 1.

FIG. 8 is a schematic diagram of an alarm system according to the invention that can accommodate to different communication standards.

FIGS. 9 and 10 are electrical schematic diagrams of one feature of the invention.

FIGS. 11-17 are representative stylized screenshots of the type that could be produced by the invention on a display panel such as that of FIG. 1.

### DETAILED DESCRIPTION

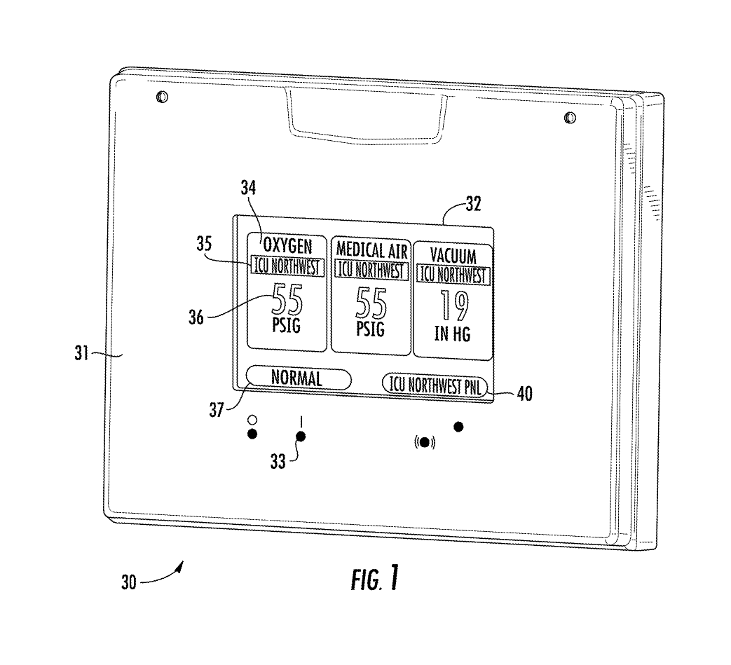

FIG. 1 is a front perspective view of an alarm panel according to one embodiment of the invention. The alarm panel is broadly designated at 30 and includes a housing 31 and a display 32. Several additional indicators e.g. LED 33 are positioned near the display 32, for supplemental purposes. Details about the display 32 and its relationships and use with other elements will be discussed with respect to the other Figures, but with respect to FIG. 1 the display 32 has been preprogrammed to display a monitored location, the identity of two medical gases (and of medical vacuum), the relative status of those gases (“low”), the actual pressure of the gases, and the status of the vacuum. Other parts of the display provide text messages that the gases are in a normal condition and repeat the identity of the zone and thus the identity of the panel. The manner in which the alarm system provides these badges and information will be discussed with respect to certain of the other Figures.

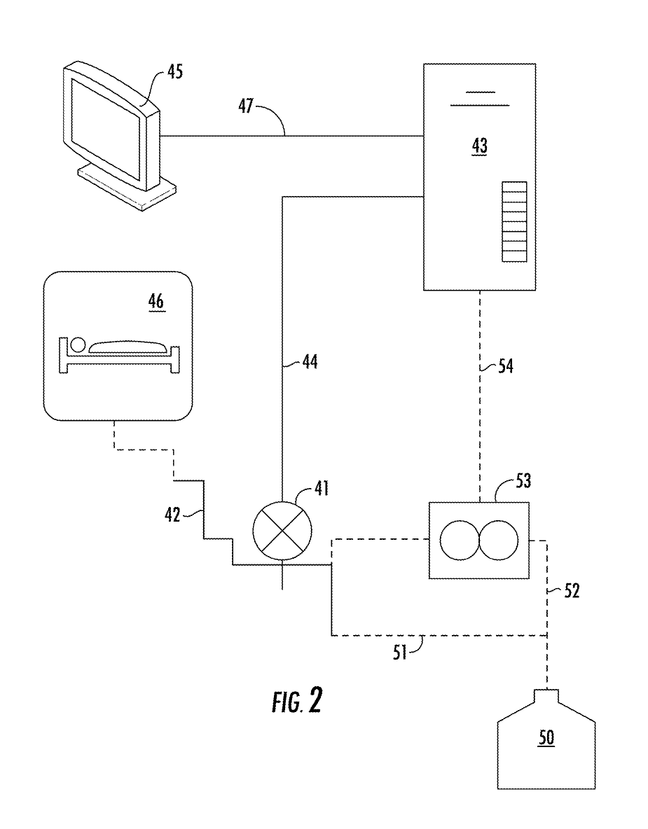

FIG. 2 illustrates a method embodiment of the invention, and in particular a method of moderating the gas supply in a medical system in the (e.g.) hospital environment. The method comprises monitoring a characteristic of the system or of a gas (in this context a medical gas or vacuum) in the system using at least one monitoring instrument (illustrated here as the gas pressure gauge 41) positioned in a medical gas supply network symbolically illustrated by the solid line 42. A signal is generated by, and sent from, a monitoring instrument to a processor 43 (alternatively referred to herein as a “CPU”) over an appropriate communication system 44 when the characteristic measured by the monitoring instrument passes a predetermined threshold. A master alarm monitors medical gas and vacuum source equipment and main pipelines. An area alarm panel monitors medical gas and vacuum systems serving a specific area. For example, if pressure is being monitored, the particular signal is sent when the gas pressure drops below the threshold pressure. As discussed herein, characteristics such as gas volume (in the form of flow rate) and temperature can be monitored, as well as the status of electrical and mechanical items such as pumps, compressors, manifolds, switches and relays, particularly where such status has consequences for the medical gas network.

When the CPU 43 receives a particular threshold signal from the monitoring instrument 41, the CPU 43 generates a fault signal (also referred to as an “alarm”). The CPU 43 uses the fault signal to retrieve a stored message (also referred to as an “instruction”) in response to the fault signal. In particular, the stored message is other than the fault or threshold condition monitored by the instrument. This CPU 43 sends the stored message to an output illustrated as the display 45 which is part of a medical gas alarm module such as illustrated in FIG. 1. In many embodiments, the display 45 is a touch-based input-output screen of the type found on inter alia cellular telephones, tablet computers, some laptop computers, and some desktop computers. The display has a color capability, and is usually as flat (thin) as possible. Current examples include liquid crystal display (LCD) and light emitting diode (LED) monitors.

Stated differently, the alarm system will provide two different outputs in two separate steps. The first is the fault signal for an “alarm” indicating the basic problem, and the second is the stored message(“instruction”)—which is different from the fault signal—and provides in many cases instructions for dealing with the condition that generated the original fault signal.

Because the display 45 gives an indication other than the output from (for example) the pressure gauge 41, it provides the capacity to visibly present a customized plan of action, or notify a desired or necessary party, or a general plan of action to be carried out in response to an alarm condition. In current embodiments the display can be customized to provide up to 72 different messages on the screen interface.

As set forth in the background, conventional alarms merely indicate the condition, and fail to offer any further information or helpful course of conduct. The capability of the invention to provide customized messages in response to the measured conditions gives it a significant functional advantage over such conventional medical alarms.

FIG. 1 also illustrates some elements that are described in further detail with respect to additional embodiments. These include the graphical designation for a patient room 46 which the skilled person will understand could also represent an intensive care unit, a surgical suite, or any other area of a hospital or other medical facility. The skilled person also understand that as symbolized by the patient room 46, the alarm can respond to a number of rooms in a zone and is not limited to a single room application.

As some other details, FIG. 2 illustrates a schematic communications line 47 between the CPU 43 and the monitor 45, a gas supply graphically designated at 50, supply lines 51 and 52, one of which potentially includes a flow meter 53 which is likewise potentially connected to the CPU 43 through the communications line 54. The basic characteristics of a gas are, of course, its identity, its pressure, its volume (measured here as flow rate) and its temperature. Thus, as set forth in the other Figures and with respect to other embodiments, a thermometer, thermocouple or other temperature sensor could be included along with, or in place of the pressure gauge 41 or the flow meter 53 if desired or necessary. Other sensors such as hygrometers can be included where desired or necessary.

In the hospital context, the performance of the equipment is usually just as important as the particular characteristics or amounts of gases moving through the system. Thus, the invention includes monitoring the status of items such as compressors (i.e., for compressed air), manifolds that distribute gas between and among multiple sources and multiple destinations, and vacuum pumps and their associated equipment. The system can provide desired information about any or all of these. Examples include (but are not limited to) a signal when a manifold switches between a primary gas supply and a secondary gas supply; a signal when a liquid gas supply is switched to a bottled gas back-up; a signal when a reserve supply is in use, and a signal when a reserve supply is low.

In the hospital context, a problem at the source of the gas or vacuum or compressed air usually will become evident everywhere, but a problem at a particular area is usually local in scope.

As set forth with respect to several of the embodiments, the method can comprise the step of sending the stored message to the monitor 45 or another destination such as an Ethernet protocol network, a Wi-Fi transmitter (e.g., the 802.11 standards), an email server, addresses on the Internet from which the message can be accessed on demand, and combinations of these outputs.

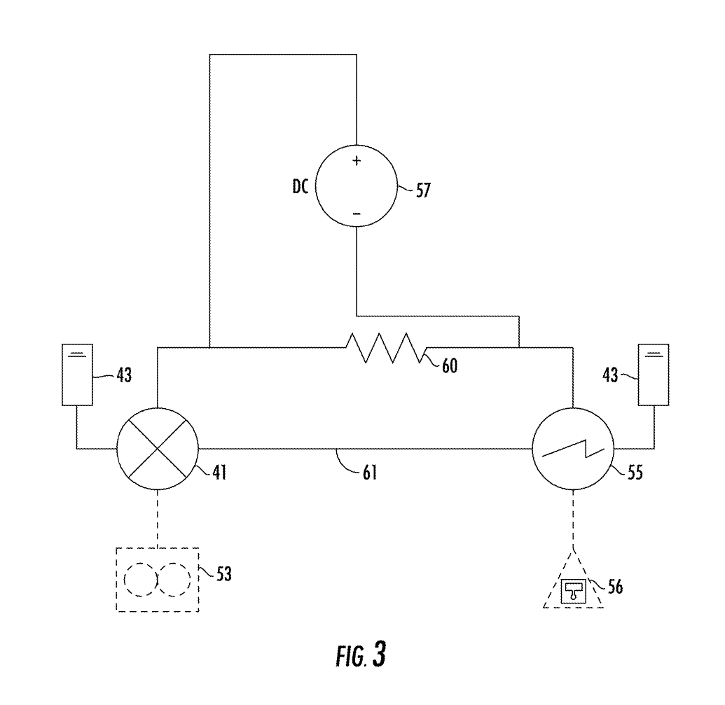

FIG. 3 is a schematic diagram of another embodiment according to the invention. In this embodiment, the medical gas alarm system includes a gas sensor, again illustrated as the pressure gauge 41 which is positioned or arranged to measure a characteristic of a medical gas in a medical supply network. A programmable monitor illustrated as the indicator 55 displays the gas characteristic from the gas sensor 41. A current loop (e.g., 4-20 mA) between the gas sensor 41 and the monitor 55 permits a desired output to be calibrated on the monitor 55 using the 4-20 mA current range. The most common current signal standard in modern use is the 4 to 20 milliamp (4-20 mA) loop, with 4 milliamps representing 0 percent of measurement, 20 milliamps representing 100 percent, 12 milliamps representing 50 percent, and so on. A convenient feature of the 4-20 mA standard is its ease of signal conversion to 1-5 volt indicating instruments. A simple 250 ohm precision resistor connected in series with the circuit will produce 1 volt of drop at 4 milliamps, 5 volts of drop at 20 milliamps, etc. As a result, the medical gas alarm system can be programmed to display the gas characteristic from the sensor 41 within the program range defined by the 4 mA and 20 mA current boundaries. The 4-20 mA current loop can be programmable, and in the schematic diagram the small processors (again numbered as 43) can be used to program the transmitter 55, the pressure gauge 41, or both.

Other standard analog (i.e., not digital and not otherwise binary) inputs are familiar to those of skill in this art and include 0-1V, 0-5V, 0-10V and others.

As in the first embodiment, the gas sensor can be other than a pressure gauge and thus FIG. 3 illustrates the flow meter 53 in dotted lines with the loop programmed or calibrated between two selected gas flow rates. Similarly, the loop can alternatively include a different type of alarm 56 and in which the loop can be programmed so that the alarm 56 signals at respective high and low set points. As discussed with respect to the overall system, these loops can also be used to provide the status of equipment including but not limited to respective compressors, vacuum pumps, and manifolds.

The elements and operation of a 4-20 mA loop are familiar to person skilled in the art because they are used in a similar fashion for other indicators systems. Thus the loop illustrated in FIG. 3 is exemplary rather than limiting, and includes the direct current power supply 57, the resistor 60, and the necessary wires 61.

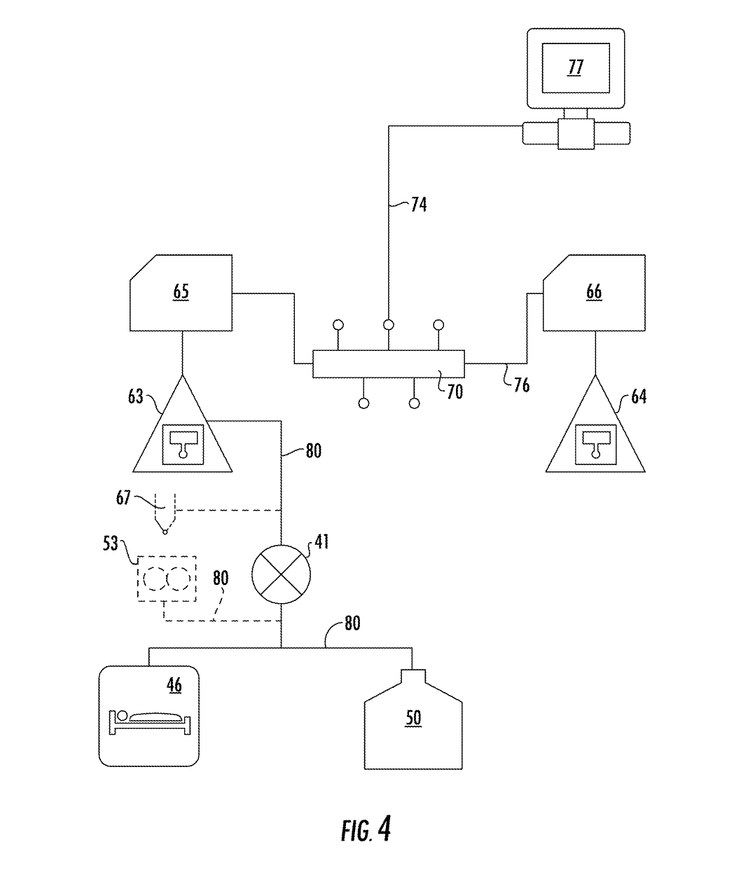

FIG. 4 is a schematic diagram of another embodiment of the invention in which a remote Ethernet solution gives users the capacity to send data points and readings over an Ethernet connection between panels. This eliminates costly wiring and from a practical standpoint, one Ethernet cable is simpler and easier to position in place (“run”) than a plurality of signal wires. Ethernet protocols, standards, and hardware are of course familiar to the skilled person.

Using the invention, when multiple alarm panels are connected on a single network, the alarm readings from one alarm can be shown on any other alarm on the network, but while avoiding the necessity of wiring all of the alarms to all of the (e.g.) sensors. For example, two alarms can be connected to the network where two of the alarm points from the first alarm can be displayed on the second alarm.

As another example (but not a limiting one) two alarms could be connected to the network so that any alarm information from one alarm will be displayed on the second alarm. Given the capability of Ethernet networks and communication, any alarm panel (display) can be programmed to show the information from any other similarly programmed panel on the network.

Accordingly, FIG. 4 illustrates a medical gas alarm system comprising at least first and second (“master-slave,” “primary-secondary”) alarm stations 63 and 64. Each station includes a respective Ethernet interface 65 and 66. The first alarm station 63 is connected to a gas monitoring instrument (or a circuit condition indicator) which again is illustrated as potentially being the thermocouple 67, the flow meter 53, or the pressure gauge 41.

The first alarm station 63 transmits information generated at or originally received by the first alarm station 63, over an available Ethernet network and using Ethernet protocol, to the second alarm system 64. In FIG. 4, the Ethernet network is indicated by the symbol 70, by the communication lines 72, 73, 74, 75 and 76, and by the network symbol 77.

As illustrated in FIG. 4, the second alarm station 64 is connected to the first alarm station over the Ethernet network and the second alarm 64 displays the information from the first alarm station (i.e., other than the Ethernet network, the second alarm 64 is not connected to any monitor, detector, gauge or switch).

The alarms 63 and 64 can include any appropriate indicator system such as indicators based on sound, lighting, and graphical user interfaces. As in the other embodiments herein, in most cases the system includes a human machine interface (HMI) with input and output capabilities, of which a touch screen is currently a helpful and exemplary embodiment.

FIG. 4 also schematically illustrates the medical gas network indicated by the gas supply 50, the patient room (or equivalent location) 46 and the piping or tubing for the network, all of which is generally designated that 80. As in other embodiments, it will be understood that the gas lines 80 that represent the medical gas network can supply a plurality of different zones or different individual locations or both.

In current embodiments, this embodiment of the invention can include between two and 12 of the secondary alarm stations such as 64.

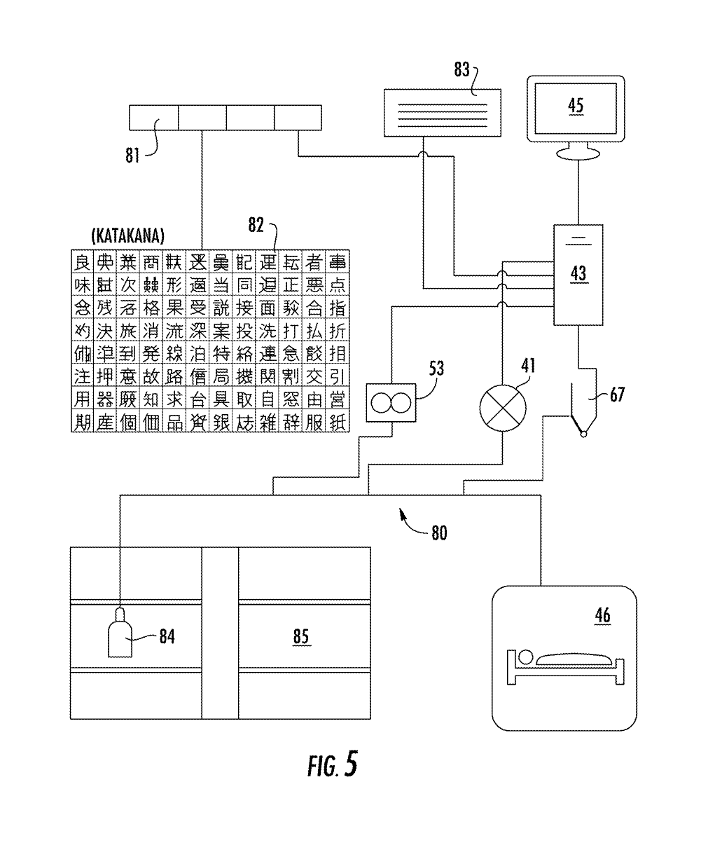

FIG. 5 is a schematic diagram of another embodiment of the invention which gives the user the capacity to input custom messages or locations into the alarm in a graphic character language (e.g. Chinese) using a native language keyboard. Images are used instead of text strings and can be created using known techniques such as HTML5 Canvas objects which can be sent to the input using the web server embedded in the alarm.

As used herein, a graphic language (or a “visual language”) is a system of communication using visual elements rather than letter strings. This is helpful because even those character languages with relatively brief alphabets (e.g., Korean) contain font sets which require large amounts of memory and a complex keyboard for creating messages.

FIG. 5 accordingly illustrates a medical gas alarm system that includes memory 81 and a set of graphic images illustrated by the Katakana character set 82. A display 45 is in communication with the memory 81, and a processor (again indicated at 43) is in communication with both the memory 81 and the display 45. In the current context, some form of digital memory is generally most useful, but it will be understood that to the extent other forms of memory can be used, the alarm system can incorporate them.

As in other embodiments, a gas monitor is again illustrated as the pressure gauge 41, the flow meter 53, or the thermocouple 67. The monitor is in communication with the processor 43. In this embodiment the processor 43 selects an image (or a plurality of images) from the memory 81 based upon the signal from one of the gas monitors, and the processor 43 then provides the image to the display 45.

In the current embodiments, the images are other than modern English characters and in many cases (but not exclusively) can be characters from the Greek alphabet, or Japanese, Chinese, Korean, Arabic, and Cyrillic (Russian) character sets. In this embodiment, the graphic images are HTML5 Canvas objects. The nature and use of HTML language, including Canvas objects is generally well understood by persons skilled in the relevant art, and will not be discussed in detail herein.

As symbolized by the keyboard 83, the user can incorporate a native language keyboard to store the message in the desired character sets in memory as an image file instead of a text or character string.

As in the earlier embodiments a gas network, the lines of which are broadly designated at 80, includes a gas supply, which in FIG. 5 is schematically illustrated as the tank 84 in a tank house 85. The gas network 80 provides gas (or vacuum) to a plurality of locations symbolized by the patient room 46. The skilled person again understands that the system usually also includes gas sources, manifolds, pumps and compressors, among other equipment.

FIG. 6 is a schematic illustration of another embodiment of the alarm system according to the invention. In this embodiment, the medical gas alarm is broadly designated at 87, and includes a web server 90 as part of the medical gas alarm and a Wi-Fi capability symbolized by the transmitter 91.

As used herein, a web server is a computer that has the capability to store, retrieve, or produce information in language that can be transmitted over the Internet, and displayed in a browser. The web server must also have a permanent Internet address (Internet protocol address). Additionally, a web server computer takes advantage of web server software which in layman's terms processes requests from all the browsers (technically called clients) and responds with the proper information, which is usually presented as a web page. Typical web server software includes an open source HTTP server software. Although the requirements for web servers are generally robust, with modern microelectronics, they can be included in the actual medical gas alarm.

As in the other embodiments, the medical gas alarm 87 is connected to one or more of the gas monitoring (or related monitoring) items schematically illustrated as the pressure gauge 41, the flow meter 53, or the thermocouple 67.

In this embodiment the alarm system includes memory symbolically illustrated at 93 as part of the alarm, or alternatively as memory 94 external to the alarm, but both in communication with the web server 90.

In this embodiment, by connecting one additional circuit board or equivalent item inside the alarm 87, the alarm 87 has the Wi-Fi capability. The Wi-Fi capability gives the user the ability to download event files and view items from the web server 90 built into the alarm panel. As further schematically illustrated in FIG. 6, the information that originates at the gas monitor eventually is produced individually or collectively as a webpage on a browser 95 using a computer 96 that communicates with the alarm 87 only through the Wi-Fi capability.

For the sake of completeness, FIG. 6 also schematically illustrates the medical gas network 80, the symbolic patient room 46, and a plurality of gas tanks 84 representing the central source (or even a partial or localized gas source).

The capabilities of the elements described herein also provide the capacity for better methods of monitoring medical gas systems. Thus, in another aspect, the invention is a method of monitoring the status of a medical gas system by repeatedly measuring a characteristic of the medical gas distribution network using a sensor positioned as part of the distribution network, and doing so for a defined time interval.

The characteristics measured by the sensor during the defined time interval are sent from the sensor to digital memory (or its equivalent) in which the measurements can be stored and from which the indexed measurements can be retrieved. Thereafter groups of the stored and indexed measurements can be periodically retrieved based upon the designated time interval (usually days) and sending those groups to a processor. The processor can then generate a report from the retrieved groups and produce the report in a form substantially compliant with a licensing or accreditation protocol.

This aspect also illustrates that the monitoring step can include monitoring a plurality of characteristics of a single gas in the network, or monitoring a single characteristic of a plurality of gases in the network, or monitoring a plurality of characteristics of a plurality of gases in the network, or monitoring the status of a gas source (supply) or compressors, vacuum pumps, manifolds, or any other relevant items.

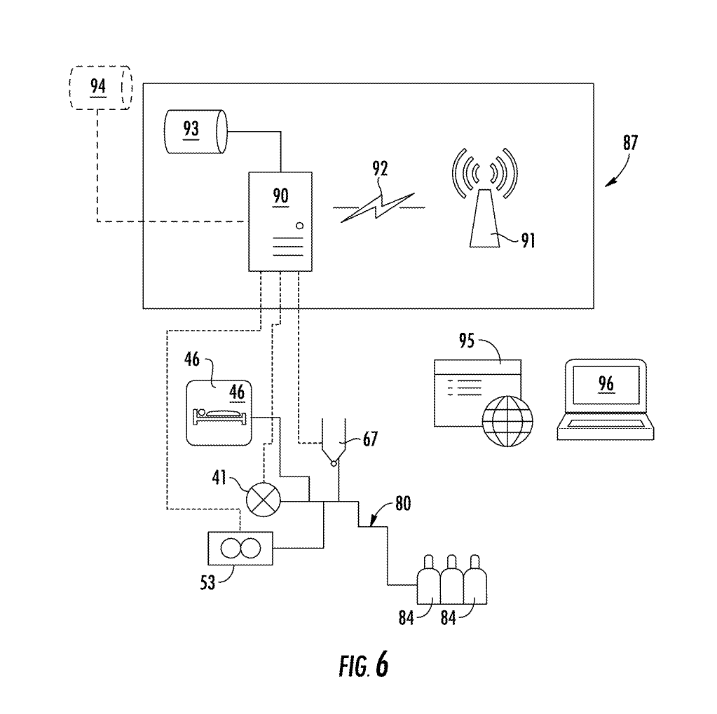

FIG. 7 illustrates another aspect of a medical gas alarm system that includes a sensor connected to a medical gas network. For the sake of clarity, a gas network per se is not illustrated in FIG. 7, but the potential sensors shown as the flow meter 53, the pressure gauge 41, and the thermocouple 67, are illustrated and connected to the alarm 56. A processor 43 is connected to the alarm 56, and a human machine interface, typically a touch screen display with input and output capabilities, is connected to the processor 43. The processor 43 either includes or is connected to appropriate memory 81 and the memory 81 includes one or more databases of (for example) text 100, color 101, and line or graphic design 102 that can be applied to the human machine interface and to the output information that the interface provides based upon the sensor 56 and the processor 43.

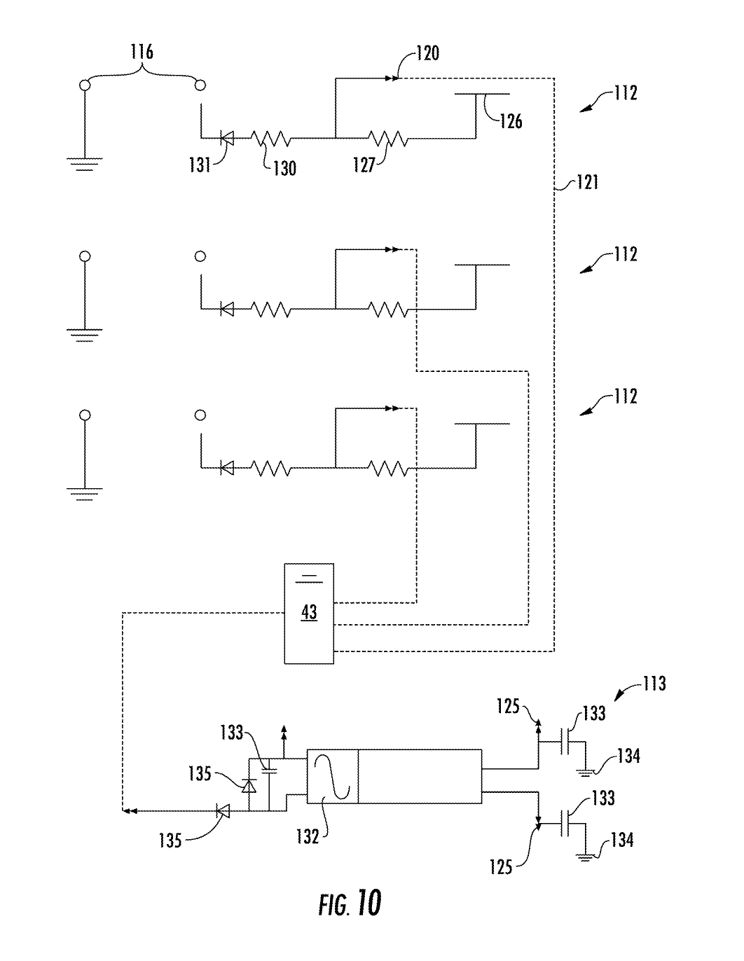

FIGS. 11-17 and their associated text provide more detail about these features.

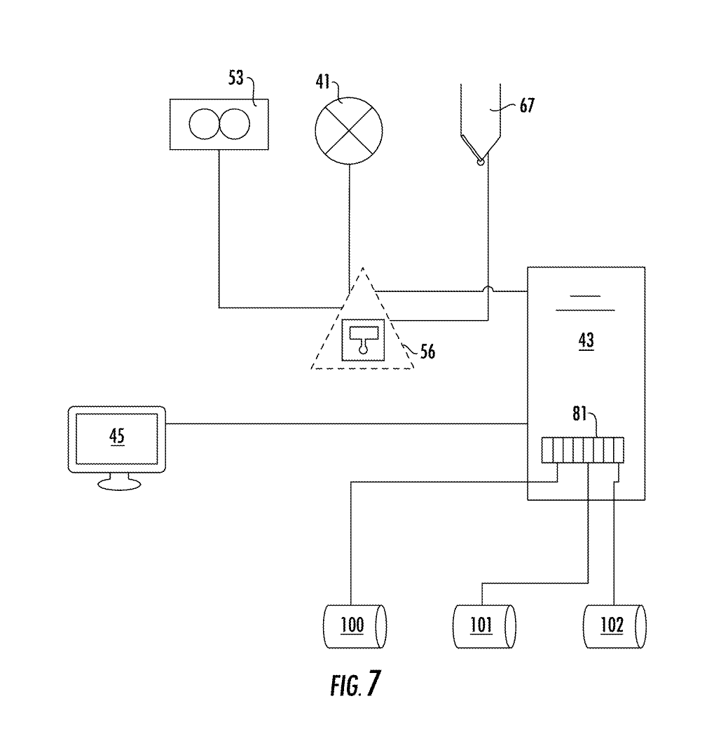

FIG. 8 is a schematic view of another aspect of the invention that provides the capability to have the alarm communicate using two or more communication standards. As a background example, the United Kingdom uses two standards for communication between alarms in medical facilities (“Shirer” and “Medipoint”). Both are based on serial data communication, but use different signal voltages and different data rates. This tends to limit medical facilities to one type of standard equipment or the other, but generally both cannot be included in any convenient fashion.

The invention provides at least two interfaces to the alarm circuit board so that the alarm can receive and transmit on two standards of communication. The invention also has the capability to communicate using Ethernet protocol which in turn provides the capability to concurrently receive signals from the two different types of communication and send the information over the Internet to a desired destination such as a building automation system, or a remote alarm panel. Similarly, the alarm can receive more than one type of communication as well as communication over the Internet and can display signals from all three sources.

Thus, FIG. 8 illustrates a medical gas alarm broadly designated at 103 that includes a first communication interface 104 and a second communication interface 105. The first communication interface 104 uses a different signal voltage and a different data rate than the second communication interface 105.

The alarm also includes an Ethernet interface and an Ethernet network which is again indicated at 65 and 70 consistent with FIG. 4. Both the first and second communication interfaces 104 and 105 join the Ethernet network along the indicated communication lines, all of which are labeled at 106 for clarity. Depending upon configuration, the interfaces 104 and 105 can also communicate externally with the network symbolized at 77 using the external communication lines 107. The interface 104 can communicate externally with a secondary alarm which uses the 104 standard of communication using communication line 107. The interface 105 can communicate externally with a secondary alarm which uses the 105 standard of communication using the communication line 108.

The network 77 is in turn connected to any number of items, two of which are schematically suggested in FIG. 8 as a building automation system 110 or other remote alarm panels 111.

FIG. 8 also illustrates the same suggested gas monitors as in previous Figures; i.e. the pressure gauge 41, the flow meter 53, and the thermocouple 67.

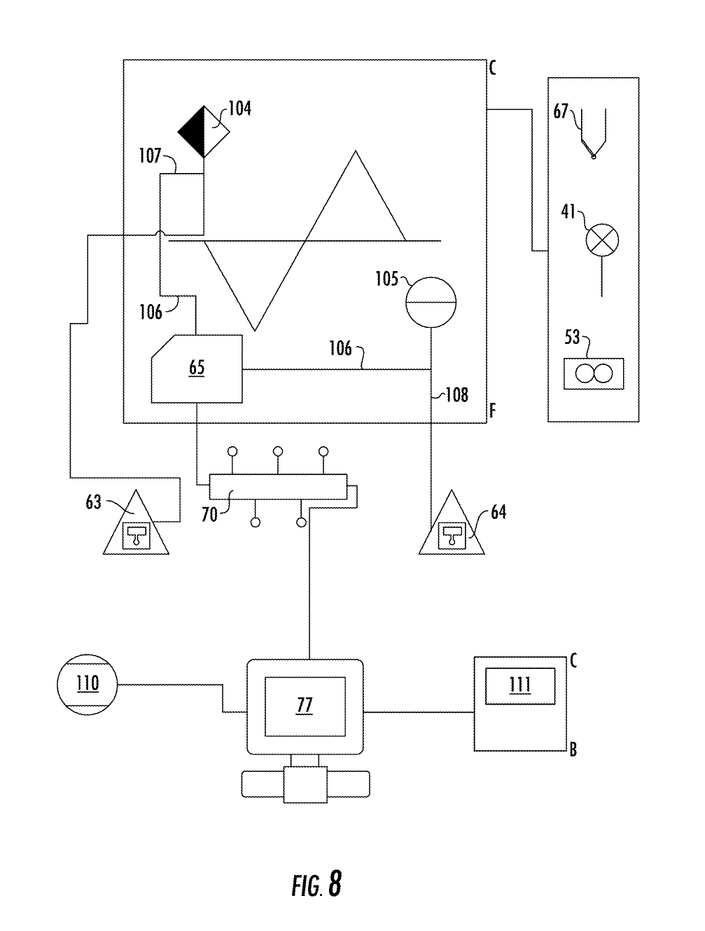

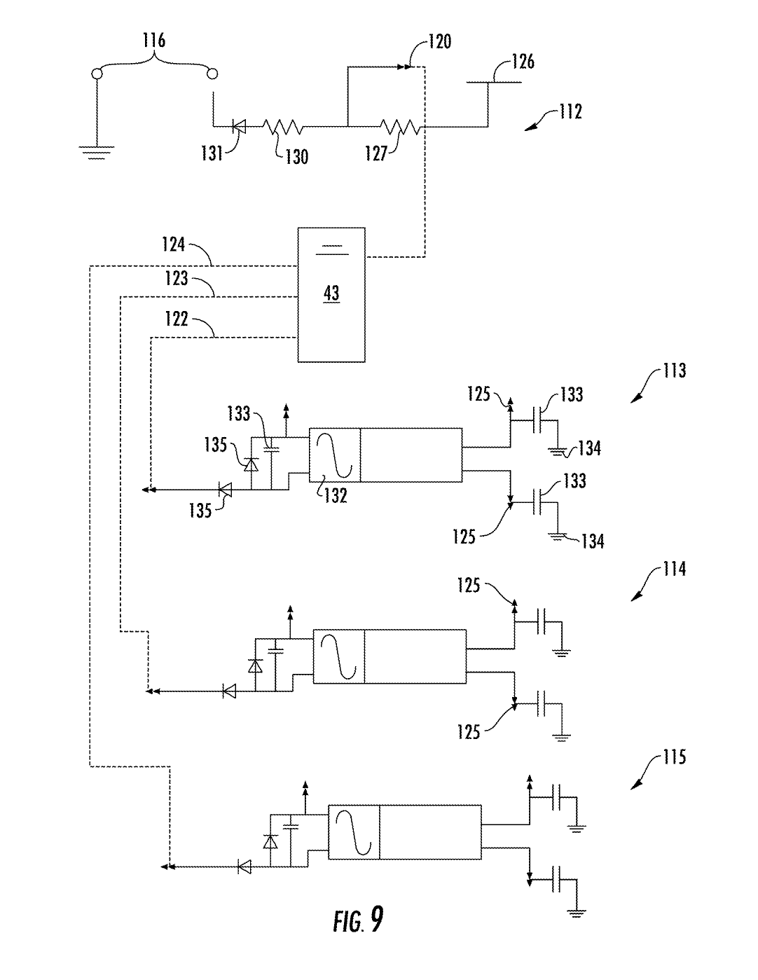

FIGS. 9 and 10 illustrate another aspect of the invention in which signals from one source can be sent to many alarm panels, or in which signals from many sources can be directed to one alarm panel.

Conventionally, alarms are used on a one-to-one ratio with signal inputs and relay outputs. In other words, one relay output is used indicate the condition of one signal input. In order to use multiple relay outputs, conventional alarms must “jump” the signal on to a second input and use that second input as well. Additionally, if a user seeks to run one relay from multiple inputs, the user is required to run the signal through more than one contact point.

Using the invention, a user can wire multiple signals from a piece of equipment to the signal input board and then use only one relay for all of the signals. If any of the conditions are bad, the relay output will open.

This also provides the capability for a user to have a duplicate output relay for a single signal, and two output relays can be selected to open when the condition for one signal is bad.

This capability reduces the number relays needed in an alarm panel and gives the user the capacity to make changes without having to install or move wires, which typically would require both a licensed electrician, and a recertification of the relevant portion of the facility.

Accordingly, FIG. 9 shows an input single signal circuit broadly designated at 112 from source equipment that communicates with a processor, which is again designated at 43, and three signal output circuits broadly designated at 113, 114, and 115.

In FIG. 9, input from the source equipment is represented by the contacts 116 and is sent as an input signal (symbolized by the arrows 120 and the communication line 121) to the processor 43. The processor 43 can then send one or more signals to one or more of the output relays 113, 114 or 115 respectively using the communication lines 122, 123, or 124. Each of the relay circuits 113, 114, and 115 have respective outputs to (for example) a building management system, and these are indicated by the double arrows 125 (for all three relays).

In slightly more detail, the signal input circuit from the source equipment also includes a power supply symbolized by the line 126, resistors 127 and 130, and diode 131.

Each of the relay circuits 113, 114, and 115 can be substantially identical (or otherwise equivalent as persons skilled in the art may prefer). As illustrated, each relay circuit includes a relay 132, capacitors 133, ground connections 134, and diodes 135. The nature and operation of these basic components and relay circuits are well understood the art and will not be otherwise explained in detail herein. As noted previously, skilled persons can modify the circuits appropriately to obtain the same result.

FIG. 10 is very similar to FIG. 9, but illustrates the use of multiple input circuits to the processor 43 and a single relay output. Accordingly the elements are essentially identical to those in FIG. 9 other than their arrangement, and the elements of FIG. 10 are number accordingly for consistency.

Using these circuits, the microprocessor can assess the state of the input signal or signals and control the output relay or relays independently. The user can select those outputs to be affected by selected inputs. As an example, if a particular piece of source equipment has five relay outputs, the facility users have the capacity to see these outputs independently on a master alarm. At the same time, if the building management system (“BMS”) needs only to know if any of the relays are opened, the master alarm can map the input signals to the single desired relay output.

As a result, the user can wire fewer points in the alarm (reducing the possibility for error) and run less wiring through the walls (thus providing cost savings).

FIGS. 11-17 are representations of screenshots of the type that can be produced using the medical gas alarm system of the invention. The screenshots can be produced based on several input options as previously described; e.g., a hard-wired or Bluetooth keyboard, a WiFi connection, or (most conveniently) a touch screen on the panel itself that provides both input and output capabilities. Of course, the alarm system of the invention can take advantage of some or all of these options rather than being limited to one of them.

FIG. 11 illustrates a home or primary screen from which the alarm system can be programmed for various purposes in the manners set forth previously.

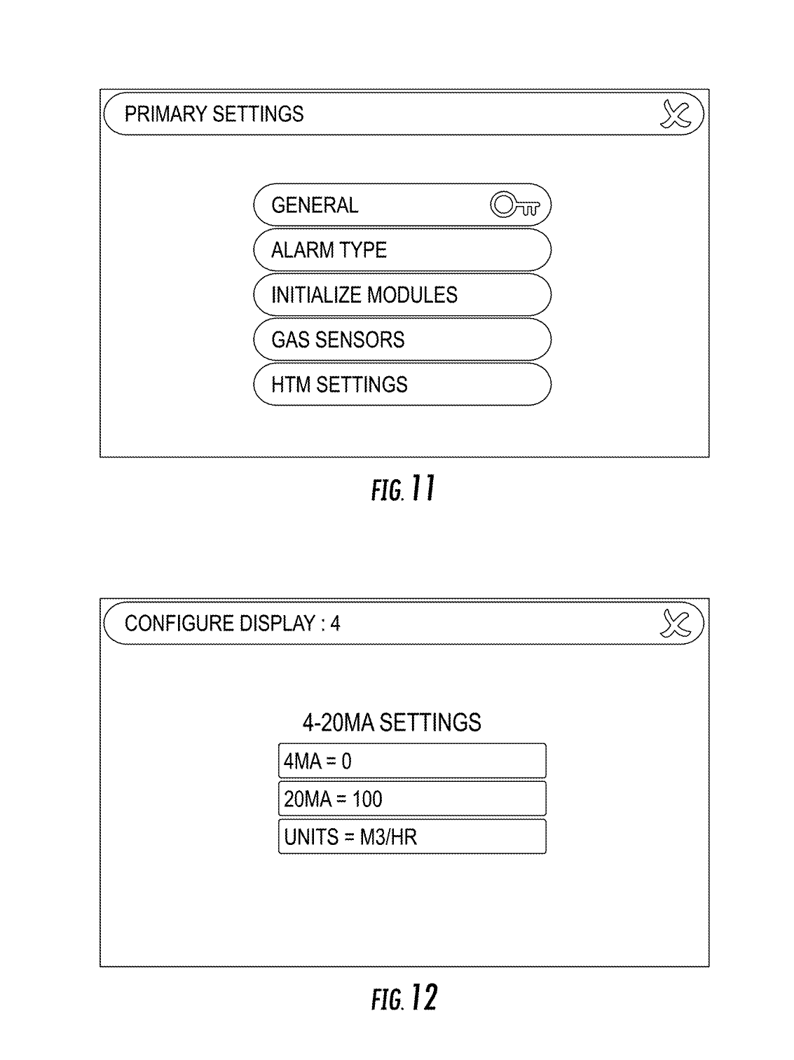

FIG. 12 illustrates a screenshot in which the input/output display is configured so that the display will produce a desired output from a 4 mA-20 mA loop. In FIG. 12, the display is presenting the output of a flow meter, with the display being set for units of cubic meters per hour (“M3/HR”), and with the 4 mA-20 mA range for these particular units being set between 0 and 100. As set forth earlier, however, the advantage of the loop is that it could just as easily be programmed to present a smaller or larger range.

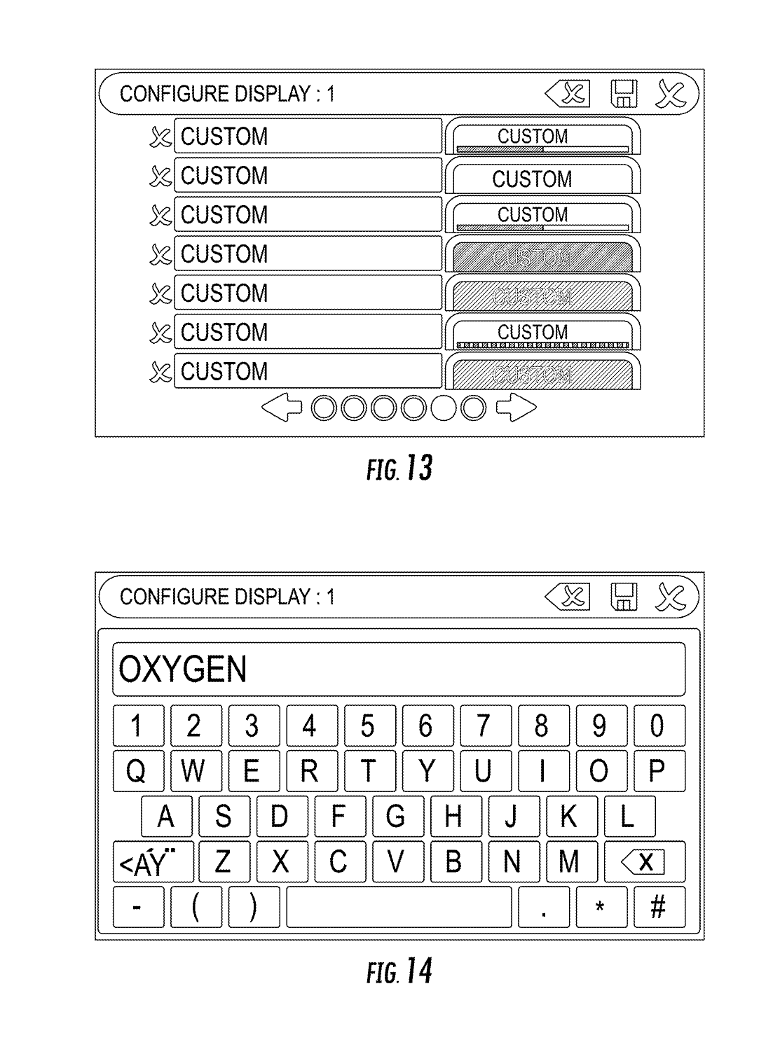

FIGS. 13 and 14 show the manner in which the badges displayed at the alarm panel (e.g. FIG. 1) can be programmed using the human machine interface. FIG. 13 illustrates that a plurality of badges can be programmed on each screen (seven are illustrated), but this is a limitation of the screen size rather than of the invention. FIG. 14 further illustrates that a virtual keyboard can be produced on the touchscreen or other input, so that the badges can be customized and configured directly from the touchscreen.

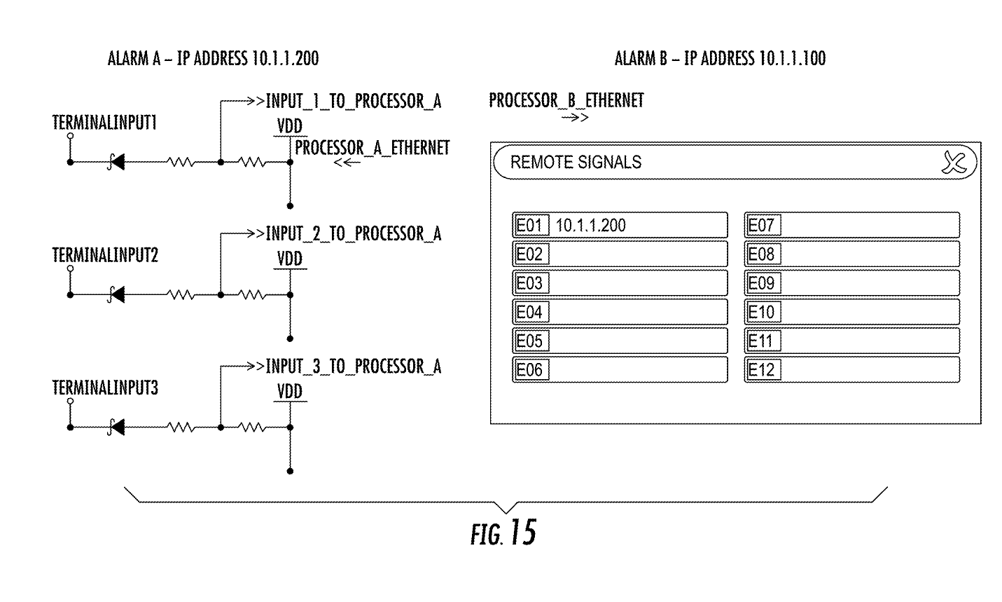

FIG. 15 is related to FIGS. 9 and 10, and shows (again) the multiple input relays corresponding to FIG. 10 and the manner in which they can be programmed from the interface and in particular from an input/output touchscreen.

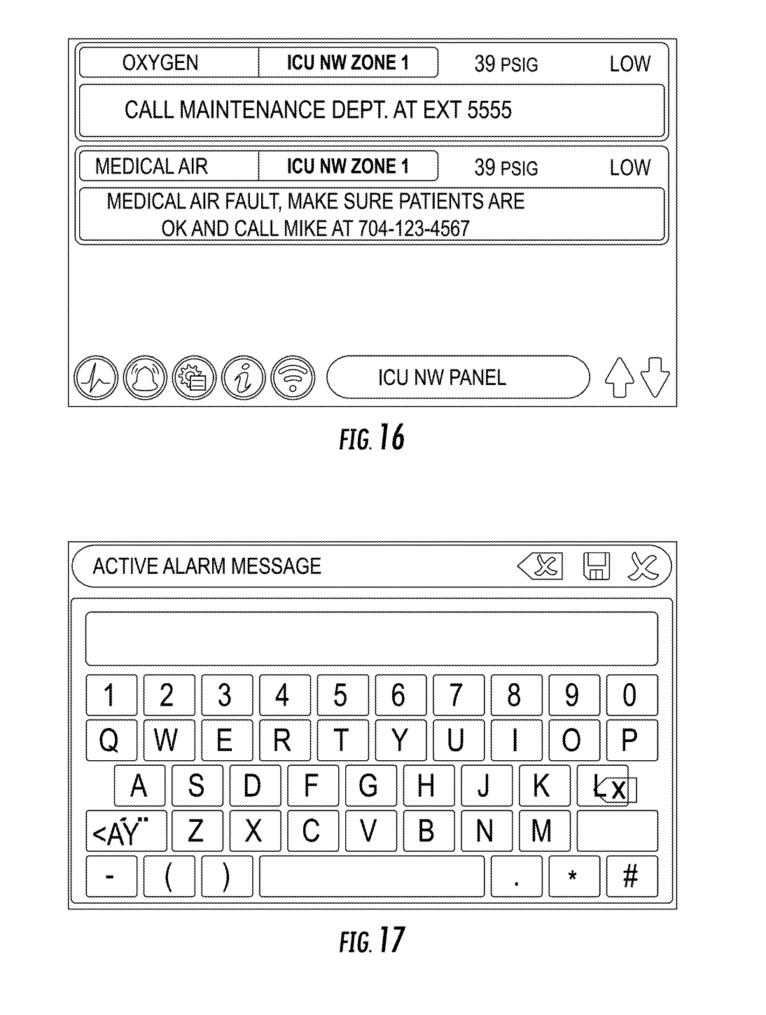

FIGS. 16 and 17 illustrate the customized output that the panel can display based upon the components of the invention. FIG. 16 illustrates that of the gases being monitored, oxygen and medical air are each in a low-pressure state in a particular identified zone. More importantly, however, FIG. 16 illustrates that the panel display shows the actual pressure of each gas and—perhaps most importantly—the custom message in response to the pressure that can be retrieved from memory and then displayed for the user.

It will be understood that in the absence of the invention's capability, the necessary responsive activity (calling maintenance or calling “Mike” in FIG. 16) must either be known to a human user with “institutional memory” of the required steps, or would alternatively be stored in some other format, such as a paper notebook. In contrast, in an emergency the invention provides any person (employee, patient, or even a visitor) with the best information as to the required responsive activity.

FIG. 17 again illustrates a virtual keyboard which can be used to draft and then store these customized messages.

In the drawings and specification there have been set forth exemplary embodiments of the invention, and although specific terms have been employed, they are used in a generic and descriptive sense only and not for purposes of limitation, the scope of the invention being defined in the claims.

1. A medical gas alarm system comprising:
at least first and second alarm stations;
an Ethernet interface for each station;
said first alarm station being connected to a gas monitoring instrument in a medical gas network;
wherein said first alarm station transmits information generated at or originally received at said first alarm station using Ethernet protocol over an available Ethernet network to said second alarm system; and
wherein said second alarm station is connected to said first alarm station over the Ethernet network and said second alarm station displays the information from said first alarm station.

2. A medical gas alarm system according to claim 1 wherein said gas monitoring instrument is selected from the group consisting of pressure gauges, flow meters, scales, and thermometers; and
wherein said medical gas network includes:
a bank gas supply;
a plurality of gas lines fed by said bank gas supply; and
a plurality of delivery locations fed by different portions of said gas lines.

3. A medical gas alarm system according to claim 1 wherein each said alarm includes an indicator system selected from the group consisting of sound, lighting, and graphical user interfaces.

4. A medical gas alarm system according to claim 3 wherein each said alarm includes a human machine interface with input and output capabilities.

5. A medical gas alarm system according to claim 4 comprising at least 30 permutations of text, color, lines, and designs that can be applied to the human machine interface.

6. A medical gas alarm system according to claim 5 wherein said human machine interface is a touch screen.

7. In a medical gas alarm system, the improvement comprising:
a Web Server in the medical gas alarm; and
a WiFi circuit in said medical gas alarm and in communication with said Web Server.

8. A medical gas alarm system according to claim 7 wherein said medical gas alarm is selected from the group consisting of a pressure sensor, flowmeter and temperature sensors and is connected to a gas sensor in a medical gas network.

9. A medical gas alarm comprising:
respective first and second communication interfaces from which a medical gas alarm can both receive and transmit;
said first communication interface using a different signal voltage and a different data rate than said second communication interface.

10. A medical gas alarm according to claim 9 further comprising:
an Ethernet protocol interface so that said alarm can send information from either of said first or second communication interface to a network external to said medical gas alarm.

11. A medical gas alarm according to claim 10 connected through said Ethernet protocol interface to a hospital network external to said medical gas alarm.

12. A medical gas alarm according to claim 11 wherein said hospital network includes items selected from the group consisting of a building automation systems and remote alarm panels.
